(19) United States
(12) Patent Application Publication
(10) Pub. No.: US 2011/0020326 A1
(43) Pub. Date: Jan. 27, 2011

(54) METHOD OF TREATING POLYCYSTIC KIDNEY DISEASE

(75) Inventors: Jordan A. Kreidberg, Wayland, MA (US); Shan Qin, Brighton, MA (US)

Correspondence Address: DAVID S. RESNICK, NIXON PEABODY LLP, 100 SUMMER STREET, BOSTON, MA, 02110-2131, US

(73) Assignee: CHILDREN'S MEDICAL CENTER CORPORATION, Boston, MA (US)

(21) Appl. No.: 12/920,652

(22) PCT Filed: Mar. 4, 2009
(86) PCT No.: PCT/US09/35972

§ 371 Date: Sep. 2, 2010

Related U.S. Application Data
(60) Provisional application No. 61/033,560, filed on Mar. 4, 2008.
(60) Provisional application No. 61/089,959, filed on Aug. 19, 2008.
(60) Provisional application No. 61/120,745, filed on Dec. 8, 2008.

(51) Int. Cl. A61K 39/395; A61P 13/12

(52) U.S. Cl. 424/130.1

(57) ABSTRACT

The present invention is directed toward methods for treating, inhibiting the progression of or eradicating polycystic kidney disease in a mammal in need thereof by providing a cMET inhibitor.

### CROSS REFERENCE TO RELATED APPLICATION

This application claims benefit under 35 U.S.C. §119(e) of the U.S. Provisional Application No. 61/033,560 filed Mar. 4, 2008, and U.S. Provisional Application No. 61/089,959 filed Aug. 19, 2008, and U.S. Provisional Application No. 61/120,745 filed Dec. 8, 2008, the contents of which are incorporated herein by reference in its entirety.

The subject matter of this application was made with support from the United States Government under NIH, Grant No. 5P50DK074030. The U.S. Government has certain rights.

### FIELD OF THE INVENTION

This invention relates to methods for treating, inhibiting the progression of, or eradicating polycystic kidney disease in a mammal in need thereof by providing a cMET inhibitor.

### BACKGROUND OF THE INVENTION

Polycystic kidney disease (PKD) is a subset of renal cystic disorders in which cysts are distributed throughout the cortex and medulla of both kidneys. PKD is usually the hallmark of a unique autosomal dominant (autosomal dominant polycystic kidney disease, ADPKD) or autosomal recessive (autosomal recessive polycystic kidney disease, ARPKD) disorder but may also be found in association with a variety of clinical conditions or acquired at some point of life by a patient with an underlying, noncystic renal disease. PKD is the most prevalent hereditary renal disorder, accounting for over 5 percent of patients on chronic hemodialysis.

ADPKD, the most common dominantly inherited kidney disease usually appears in midlife, and is characterized morphologically be massive cyst enlargement, moderate interstitial infiltration with mononuclear cells, and extensive fibrosis. Characteristic symptoms include proteinuria, abdominal pain, and palpable kidneys, followed by hematuria, hypertension, pyuria, uremia, and calculi. In about 15% of patients, death is due to cerebral aneurysm. ADPKD is caused by mutations in one of three genes: PKD1 on chromosome 16 accounts for approximately 85% of cases whereas PKD2 on chromosome 4 accounts for approximately 15%. Mutations in the so far unmapped PKD3 gene are rare. (Reeders et al., Nature 317:542-544 (1985); Kimberling et al., Genomics 18:467-472 (1993); Daoust et al., Genomics 25:733-736 (1995); Koptides et al., Hum. Mol. Genet. 8:509-513 (1999)).

ARPKD is a rare inherited disorder which usually becomes clinically manifest in early childhood, although presentation of ARPKD at later ages an survival into adulthood have also been observed in many cases. ARPKD was first studied in C57BL/6J mice in which it arises spontaneously (Preminger et al., J. Urol. 127:556-560 (1982)). The cpk mutation characteristic of this disease has been mapped to mouse chromosome 12 (Davis son et al., Genomics 9:778-781 (1991)). The gene responsible for ARPKD in humans has been mapped to chromosome 6 p. More recently, fine mapping of the autosomal recessive polycystic kidney disease locus (PKHD1) has been reported (Mucher et al., Genomics 48:40-45 (1998)).

Autosomal dominant polycystic kidney disease (ADPKD), also called adult-onset polycystic kidney disease, is one of the most common hereditary disorders in humans, affecting approximately one individual in a thousand. The prevalence in the United States is greater than 500,000, with 6,000 to 7,000 new cases detected yearly (Striker et al., Am. J. Nephrol. 6:161-164, 1986; Iglesias et al., Am. J. Kid. Dis. 2:630-639, 1983). The disease is considered to be a systemic disorder, characterized by cyst formation in the ductal organs such as kidney, liver, and pancreas, as well as by gastrointestinal, cardiovascular, and musculoskeletal abnormalities, including colonic diverticulitis, berry aneurysms, hernias, and mitral valve prolapse (Gabow et al., Adv. Nephrol. 18:19-32, 1989; Gabow, New Eng. J. Med. 329:332-342, 1993).

The most prevalent and obvious symptom of ADPKD is the formation of kidney cysts, which result in grossly enlarged kidneys and a decrease in renal-concentrating ability. In approximately half of ADPKD patients, the disease progresses to end-stage renal disease, and ADPKD is responsible for 4-8% of the renal dialysis and transplantation cases in the United States and Europe (Proc. Eur. Dialysis and Transplant Assn., Robinson and Hawkins, eds., 17:20, 1981).

Polycystic kidney disease (PKD) is one of the most common inherited disorders that result in severe and debilitating disease. There are two predisposing loci, PKD1 and PKD2, residing on chromosomes 16 and 4, respectively^{1,2}, that encode polycystin-1 and polycystin-2. Extensive study of polycystins and associated proteins has begun to elucidate the molecular biology of cystogenesis³. Nevertheless, the precise molecular mechanisms of cysts formation remain to be determined.

Several primary pathogenetic mechanisms have been considered to be responsible for cyst formation, including: 1) Abnormal regulation of epithelial cell proliferation⁴⁻⁶; 2) Abnormal trans-epithelial transport resulting in fluid accumulation in tubular lumina^{7,8}; and 3) Remodeling of the extracellular matrix (ECM), leading to abnormal epithelial morphology, proliferation and/or survival⁹⁻¹¹. Several signal transduction pathways are known to regulate epithelial cell expansion during kidney development, including those downstream of c-Ret¹², and of receptors for FGFs^{13,14} and BMPs¹³. An additional receptor tyrosine kinase, c-MET, is expressed in collecting duct epithelial cells and binds hepatocyte growth factor (HGF). Targeted mutagenesis of the c-MET or HGF genes failed to show a phenotype in the developing kidney, possibly due to liver-related embryonic lethality while the kidney is still in its early stages of development.

Integrin receptors are heterodimeric transmembrane proteins, which mediate attachment of cells to the extracellular matrix (ECM). We previously demonstrated a role for α3β1-integrin in kidney development; targeted mutation of the α3-integrin gene results in shorter and decreased number of collecting ducts in mutant kidneys¹⁸, an observation consistent with decreased branching morphogenesis and/or decreased epithelial tubule expansion. Small cysts are also observed in α3-integrin mutant kidneys, suggesting α3β1-integrin may have a role in maintaining normal tubular morphology, and dysfunction of α3β1-integrin may relate to cystogenesis. Consistent with this finding, a hypomorphic mutation in the mouse α5 laminin gene, which encodes the major ligand for α3β1-integrin, causes a phenotype that resembles polycystic kidney disease¹⁹. A major signaling pathway through which integrins regulate epithelial cell behavior involves phosphatidyl inositol-3-kinase (PI3K) and Akt^{20,21}. mTOR is a major target of Akt, and increased activation of mTOR has been suggested to contribute to cyst formation in mice and humans²². How mTOR activity is controlled in PKD is not fully understood.

The present invention is directed to overcoming these and other deficiencies in the art.

### SUMMARY OF THE INVENTION

One aspect of the present invention is directed toward a method for treating polycystic kidney disease in a subject in need thereof. The method includes providing to the subject an effective amount of a cMET inhibitor or a pharmaceutical salt thereof.

Another aspect of the present invention is directed toward a method for treatment of polycystic kidney disease. The method includes selecting a subject having polycystic kidney disease and administering to the subject an effective amount of a pharmaceutical composition comprising a cMET inhibitor.

Another aspect of the present invention is use of a cMET inhibitor or a pharmaceutical salt thereof as a medicament for, or in the manufacture of a medicament for treating polycystic kidney disease in a subject in need thereof.

### BRIEF DESCRIPTION OF THE DRAWINGS

FIGS. 1A-D HGF stimulation causes hyperphosphorylation of mTOR (a, b) and Akt (c, d) in Pkd1^{null/null} cells. Western blots for phospho-mTOR and mTOR (A) phospho-AKT and AKT (c). Densitometry is shown in (b and d) and values for each lane are shown below each blot. (a). Serum-starved Pkd1+/+ (WT) and Pkd1^{null/null} cells (KO) were incubated with media alone (control), media containing HGF (50 ng/ml for 20 min), or c-Met inhibitor (5 mM for 40 min). mTOR is hyper-phosphorylated in HGF-stimulated Pkd1^{null/null} cells. mTOR phosphorylation is inhibited by the c-MET inhibitor. (c and d) Akt is hyper-phosphorylated in Pkd1^{null/null} cells after stimulation with HGF Akt phosphorylation was detected by phospho-Akt (Ser473) antibody. All figures are representative of a minimum of 3 consistent experiments.

FIGS. 2A-C Failure of c-MET ubiquitination in Pkd1^{null/null} cells. (2A, left) Semi-quantitative RT-PCR and (2A, right) Real-Time PCR for c-Met. 18s RNA was used as an input control (left) and for normalization of Real-Time PCR (right). Pkd1 −− referes to Pkd1^{null/null} cells or tissue in all figures. (2B) Western blot of c-Met in Pkd1 +/+ and Pkd1^{null/null} cells, or α3 integrin +/+and α3 integrin −/− cells. Extracts were prepared either before or after HGF stimulation (50 ng/ml for 30 min). Densitometric quantitation is shown in the right panel. Compared with WT cells, c-Met was present at higher baseline levels and failed to be degraded in Pkd1^{null/null} cells. GAPDH is shown as a loading control. (2C) Pkd1 +/+ and Pkd1^{null/null} cells were stimulated with HGF (as above), and cell lysates were immunoprecipitated with c-MET antibody and blotted with anti-Ubiquitin. Unstimulated cells showed little ubiquitination of c-Met. The immunoprecipitation was validated by a re-blot for c-Met.

FIGS. 3A-C (a) c-Cb1 phosphorylation after HGF stimulation is decreased in α3 integrin −/− cells and Pkd1^{null/null} cells. WT and KO cells were incubated with HGF (50 ng/ml, 10 min). Phospho-c-Cb1 and total c-Cb1 were detected by Western blot. c-Cb1 phosphorylation by HGF is weaker in both Pkd1^{null/null} and α3 integrin−/− cells, compared with their counterpart's wild type controls; (b) Inaccessibility of α3β1 integrin and c-Cb1 in Pkd1^{null/null} cells. Cells were labeled with membrane-impermeable Sulfo-NHS-Biotin, cell lysates precipitated with Avidin, and non-precipitated material re-immunoprecipitated with anti-α3β1 integrin. More α3β1 integrin is membrane-accessible in WT cells, and c-Cb1 could be co-immunoprecipitated with α3β1 integrin in WT or Pkd1^{null/null} cells; (c) c-Cb1 binds α3β1 integrin in both WT and Pkd1^{null/null} cells. Lanes are designated as starting lysate, anti-α3β1 integrin or IgG control immunoprecipitated material, and residual non-immunoprecipitated material. The membrane was reblotted with anti α3β1 integrin antibody to validate the immunoprecipitation. c-Cb1 immunoprecipitated with α3β1 integrin in both Wt and Pkd1^{null/null} cells.

FIG. 4 Discontinuous sucrose gradient enrichment of the Golgi apparatus from WT and Pkd1^{null/null} cells. Both α3β1 integrin and c-Cb1 are present in the Golgi fraction from Pkd1^{null/null} cells, but neither was detected in the Golgi fraction from WT cells. Western blot of GM130 in the lower panel validates the Golgi enrichment.

FIG. 5 Defective glycosylation of α3 integrin subunit in Pkd1^{null/null} cells. Western-Blot using an anti-α3 integrin antibody. Treatment with Endo H or PNGase designated above the lanes, the two leftmost lanes are untreated. PNGase removes all N-linked glycosylation, whereas EndoH removes only high mannose glycosylation. α3 integrin subunit shows a faster migration in Pkd1^{null/null} cells. Digestion with de-glycosylating enzymes eliminates this difference.

FIG. 6 A c-MET inhibitor decreased the size and number of cysts in an organ culture model of PKD. The genotype and treatment are noted on the left and above the panels, respectively. WT and Pkd1^{null/null} mice kidneys at E15.5 were removed from embryonic mice and put in organ culture dish, containing media with 10 mM 8-Br-cAMP. 1 day later, either 5 μM c-MET inhibitor (dissolved in DMSO) or the same amount of DMSO was added to the media. Hematoxylin & Eosin stained sections of kidneys are shown after 96 hours of culture. Cyst formation was decreased by c-MET inhibitor treatment in the Pkd1^{null/null} kidneys, with no apparent effect on nephrogenesis. These are representative of three independent experiments.

FIGS. 7A-C (A) c-Met antagonists can ameliorate the cyst formations in kidneys. E13.5 embryonic kidneys were put in organ culture along with 100 μM 8-Br-cAMP, with or without 5 μM c-MET inhibitor (SU11274, from Calbiochem). (B) E13.5 embryonic kidneys were put in organ culture along with 100 mM 8-Br-cAMP, with or without 5 μg/ml c-MET neutralizing (blocking) antibody (R&D Systems). (C) E13.5 embryonic kidneys were put in organ culture along with 100 mM 8-Br-cAMP, with or without 0.5 μM c-MET inhibitor (PHA665752, from Tocris, UK). Hematoxylin & Eosin stained sections of kidneys are shown after 96 hours of culture. Cyst formation was decreased by c-MET inhibitor or c-MET neutralizing antibody treatment in the Pkd1 null/null kidneys, with no apparent effect on nephrogenesis.

FIGS. 8A-D show slides of E18.5 embryonic kidneys fixed in 4% PFA, genotyped, and paraffin sections stained with Hematoxylin and Eosin.

### DETAILED DESCRIPTION OF THE INVENTION

### Definitions

Unless otherwise stated the following terms used in the specification and claims have the meanings discussed below:

“Pharmaceutically acceptable salt” or “pharmaceutically acceptable salt thereof” refers to those salts which retain the biological effectiveness and properties of the free bases and which are obtained by reaction with inorganic or organic acids, such as hydrochloric acid, hydrobromic acid, hydroiodic acid, sulfuric acid, nitric acid, phosphoric acid, methanesulfonic acid, ethanesulfonic acid, p-toluenesulfonic acid, salicylic acid, acetic acid, benzenesulfonic acid (besylate), benzoic acid, camphorsulfonic acid, citric acid, fumaric acid, gluconic acid, glutamic acid, isethionic acid, lactic acid, maleic acid, malic acid, mandelic acid, mucic acid, pamoic acid, pantothenic acid, succinic acid, tartaric acid, and the like.

A “pharmaceutical composition” refers to a mixture of one or more of the compounds described herein, or pharmaceutically acceptable salts or prodrugs thereof, with other chemical components, such as pharmaceutically acceptable carriers and excipients. The purpose of a pharmaceutical composition is to facilitate administration of a compound to an organism.

As used herein, a “pharmaceutically acceptable carrier” refers to a carrier or diluent that does not cause significant irritation to an organism and does not abrogate the biological activity and properties of the administered compound.

An “excipient” refers to an inert substance added to a pharmaceutical composition to further facilitate administration of a compound. Examples, without limitation, of excipients include calcium carbonate, calcium phosphate, various sugars and types of starch, cellulose derivatives (including microcrystalline cellulose), gelatin, vegetable oils, polyethylene glycols, diluents, granulating agents, lubricants, binders, disintegrating agents, and the like.

“Therapeutically effective amount” refers to that amount of the compound being administered which will relieve to some extent one or more of the symptoms of the disorder being treated. In reference to the treatment of polycystic kidney disease, a therapeutically effective amount refers to that amount which has the effect of:

(1) reducing the size of the cyst(s);

(2) inhibiting (that is, slowing to some extent, preferably stopping) cyst growth and/or,

(3) relieving to some extent (or, preferably, eliminating) one or more symptoms associated with the disorder.

“cMET inhibitor” includes, for example, compounds described in WO06/108059, WO 2006/014325, and WO 2005/030140.

Compound SU11274 is (3Z)-N-(3-chlorophenyl)-3-({3,5-dimethyl-4-[(4-methylpiperazin-1-yl)carbonyl]-1H-pyrrol-2-yl}methylene)-N-methyl-2-oxoindoline-5-sulfonamide. See Us Patent publication US2004/0204407.

Compound PHA665752 is (2R)-1-[[5-[(Z)45-[[(2,6-Dichlorophenyl)methyl]sulfony 1]-1,2-dihydro-2-oxo-3H-indo1-3-ylidenelmethyl]-2,4-dim ethyl-1H-pyrrol-3-yl]carbonyl]-2-(1-pyrrolidinylmethyl) pyrrolidone. See Christensen et al (2003) A selective small molecule inhibitor of c-Met kinase inhibits c-Met-dependent phenotypes in vitro and exhibits cytoreductive antitumour activity in vivo. Cancer Res. 63 7345. Smolen et al (2006) Amplification of MET may identify a subset of cancers with extreme sensitivity to the selective tyrosine kinase inhibitor PHA-665752. Proc. Natl. Acad. Sci. USA 103 2316. Puri et al (2007) A selective small molecule inhibitor of c-Met, PHA665752, inhibits tumorigenicity and angiogenesis in mouse lung cancer xenografts. Cancer Res. 67 3529.

Compound ARQ197 is 3-(2,3-dihydro-1H-pyrrolo[3,2,1-ij]quinolin-6-yl)-4-(1H-indol-3-yl)pyrrolidine-2,5-dione. See WO2006086484.

Compound PF-2341066 is (R)-3-[1-(2,6-dichloro-3-fluoro-phenyl)-ethoxy]-5-(1-piperidin-4-yl-1H-pyrazol-4-yl)-pyridin-2-ylamine. See Cancer Research 67, 4408-4417, May 1, 2007.

Compound NK4 is N-terminal four kringle-containing fragment of hepatocyte growth factor. See WO/2005/095449.

Compound XL880 (e.g., GSK089). See Papillary Renal Cell Carcinoma phase II trial Proc Natl Acad Sci USA. 2007 December 26; 104 (52): 20932-20937.

Compound MP 470 is N-((benzo[d][1,3]dioxol-5-yl)methyl)-4-(benzofuro[3,2-d]pyrimidin-4-yl)piperazine-1-carbothioamide. See WO2005037825.

Compound K252a is (9S,10R,12R)-2,3,9,10,11,12-Hexahydro-10-hydroxy-9-methyl-1-oxo-9,12-epoxy-1H-diindolo[1,2,3-fg:3′,2′,1′-kl]pyrrolo[3,4-i][1,6]benzodiazocine-10-carboxylic acid methyl ester. See Kase et al (1986) K-252a, a potent inhibitor of protein kinase C from microbial origin. J. Antibiot. 39 1059.

C-Met blocking antibodies are known in the art, for example see WO/2004/072117.

One aspect of the present invention is directed toward a method for treating polycystic kidney disease in a subject in need thereof. The method includes providing to the subject an effective amount of a cMET inhibitor or a pharmaceutical salt thereof.

Another aspect of the present invention is directed toward a method for treatment of polycystic kidney disease. The method includes selecting a subject having polycystic kidney disease and administering to the subject an effective amount of a pharmaceutical composition comprising a cMET inhibitor.

In certain embodiments, the cMET inhibitor is selected from the group consisting of SU112274, PHA665752, ARQ 197, PF-2341066, NK4, XL-880, MP 470, K252a, c-MET blocking antibody, and combinations thereof. In a preferred embodiment the cMET inhibitor is SU112274. In another preferred embodiment the cMET inhibitor is c-MET blocking antibody.

In certain embodiments, the subject is a mammal. In preferred embodiments the subject is a human or a feline.

Agents of the present invention can be administered orally, parenterally, for example, subcutaneously, intravenously, intramuscularly, intraperitoneally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The active agents of the present invention may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active agents may be incorporated with excipients and used in the form of tablets, capsules, elixirs, suspensions, syrups, and the like. Such compositions and preparations should contain at least 0.1% of active agent. The percentage of the agent in these compositions may, of course, be varied and may conveniently be between about 2% to about 60% of the weight of the unit. The amount of active agent in such therapeutically useful compositions is such that a suitable dosage will be obtained. Preferred compositions according to the present invention are prepared so that an oral dosage unit contains between about 1 and 250 mg of active agent.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to the active ingredient, sucrose as a sweetening agent, methyl and propylparabens as preservatives, a dye, and flavoring such as cherry or orange flavor.

These active agents may also be administered parenterally. Solutions or suspensions of these active agents can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The agents of the present invention may also be administered directly to the airways in the form of an aerosol. For use as aerosols, the agents of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean ±1%.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### Administration and Pharmaceutical Composition

A compound of the present invention or a pharmaceutically acceptable salt thereof, can be administered as such to a human patient or can be administered in pharmaceutical compositions in which the foregoing materials are mixed with suitable carriers or excipient(s). Techniques for formulation and administration of drugs may be found in “Remington's Pharmacological Sciences,” Mack Publishing Co., Easton, Pa., latest edition.

As used herein, “administer” or “administration” refers to the delivery of a compound or a pharmaceutically acceptable salt thereof or of a pharmaceutical composition containing a compound or a pharmaceutically acceptable salt thereof of this invention to an organism for the purpose of prevention or treatment of a PKD-related disorder.

Suitable routes of administration may include, without limitation, oral, rectal, transmucosal or intestinal administration or intramuscular, subcutaneous, intramedullary, intrathecal, direct intraventricular, intravenous, intravitreal, intraperitoneal, intranasal, or intraocular injections. The preferred routes of administration are oral and parenteral.

Alternatively, one may administer the compound in a local rather than systemic manner, for example, via injection of the compound directly into a solid tumor, often in a depot or sustained release formulation.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, or liquid polyethylene glycols. Stabilizers may be added in these formulations, also.

Pharmaceutical compositions which may also be used include hard gelatin capsules. As a non-limiting example, the active compound capsule oral drug product formulation may be as 50 and 200 mg dose strengths.

For administration by inhalation, the compounds for use according to the present invention are conveniently delivered in the form of an aerosol spray using a pressurized pack or a nebulizer and a suitable propellant, e.g., without limitation, dichlorodifluoromethane, trichlorofluoromethane, dichlorotetra-fluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

The compounds may also be formulated for parenteral administration, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

Pharmaceutical compositions for parenteral administration include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharamcologically acceptable oil), with ion exchange resins, or as a sparingly soluble derivative such as, without limitation, a sparingly soluble salt.

Alternatively, other delivery systems for hydrophobic pharmaceutical compounds may be employed. Liposomes and emulsions are well known examples of delivery vehicles or carriers for hydrophobic drugs. In addition, certain organic solvents such as dimethylsulfoxide also may be employed, although often at the cost of greater toxicity.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions herein also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Compounds of the invention may be provided as physiologically acceptable salts wherein the claimed compound may form the negatively or the positively charged species. Examples of salts in which the compound forms the positively charged moiety include, without limitation, quaternary ammonium (defined elsewhere herein), salts such as the hydrochloride, sulfate, carbonate, lactate, tartrate, malate, maleate, succinate wherein the nitrogen atom of the quaternary ammonium group is a nitrogen of the selected compound of this invention which has reacted with the appropriate acid. Salts in which a compound of this invention forms the negatively charged species include, without limitation, the sodium, potassium, calcium and magnesium salts formed by the reaction of a carboxylic acid group in the compound with an appropriate base (e.g. sodium hydroxide (NaOH), potassium hydroxide (KOH), Calcium hydroxide (Ca(OH)2), etc.).

Pharmaceutical compositions suitable for use in the present invention include compositions wherein the active ingredients are contained in an amount sufficient to achieve the intended purpose, e.g., the treatment or prevention of a PKD.

More specifically, a therapeutically effective amount means an amount of compound effective to prevent, alleviate or ameliorate symptoms of disease or prolong the survival of the subject being treated.

Determination of a therapeutically effective amount is well within the capability of those skilled in the art, especially in light of the detailed disclosure provided herein.

For any compound used in the methods of the invention, the therapeutically effective amount or dose can be estimated initially from cell culture assays. Then, the dosage can be formulated for use in animal models so as to achieve a circulating concentration range that includes the IC₅₀ as determined in cell culture. Such information can then be used to more accurately determine useful doses in humans.

Dosage amount and interval may be adjusted individually to provide plasma levels of the active species which are sufficient to maintain the kinase modulating effects. These plasma levels are referred to as minimal effective concentrations (MECs). The MEC will vary for each compound but can be estimated from in vitro data, e.g., the concentration necessary to achieve 50-90% inhibition of a kinase may be ascertained using the assays described herein. Dosages necessary to achieve the MEC will depend on individual characteristics and route of administration. HPLC assays or bioassays can be used to determine plasma concentrations.

Dosage intervals can also be determined using MEC value. Compounds should be administered using a regimen that maintains plasma levels above the MEC for 10-90% of the time, preferably between 30-90% and most preferably between 50-90%.

In cases of local administration or selective uptake, the effective local concentration of the drug may not be related to plasma concentration and other procedures known in the art may be employed to determine the correct dosage amount and interval.

The amount of a composition administered will, of course, be dependent on the subject being treated, the severity of the affliction, the manner of administration, the judgment of the prescribing physician, etc.

The compositions may, if desired, be presented in a pack or dispenser device, such as an FDA approved kit, which may contain one or more unit dosage forms containing the active ingredient. The pack may for example comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or of human or veterinary administration. Such notice, for example, may be of the labeling approved by the U.S. Food and Drug Administration for prescription drugs or of an approved product insert. Compositions comprising a compound of the invention formulated in a compatible pharmaceutical carrier may also be prepared, placed in an appropriate container, and labeled for treatment of an indicated condition. Suitable conditions indicated on the label may include treatment of polycystic kidney disease.

The efficacy of a given treatment for polycystic kidney disease can be determined by the skilled clinician. However, a treatment is considered “effective treatment,” as the term is used herein, if any one or all of the signs or symptoms of, as but one example, polycystic kidney disease (PKD) are altered in a beneficial manner, other clinically accepted symptoms or markers of disease are improved, or even ameliorated, e.g., by at least 10% following treatment with a c-Met inhibitor. Efficacy can also be measured by a failure of an individual to worsen as assessed by hospitalization or need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the pathogenic growth of cysts; or (2) relieving the disease, e.g., causing regression of symptoms, reducing the number of cysts in a tissue exhibiting pathology involving PKD (eg., the kidney); and (3) preventing or reducing the likelihood of the development of a PKD.

An effective amount for the treatment of a disease means that amount which, when administered to a mammal in need thereof, is sufficient to result in effective treatment as that term is defined herein, for that disease. Efficacy of an agent can be determined by assessing physical indicators of, for example PKD, such as e.g., cyst formation, growth, etc.

In some embodiments of the present invention may be defined in any of the following numbered paragraphs:
1. A method for treating polycystic kidney disease in a subject in need thereof which comprises providing to said subject an effective amount of a cMET inhibitor or a pharmaceutical salt thereof.
2. The method of paragraph 1, wherein the cMET inhibitor is selected from the group consisting of SU112274, PHA665752, ARQ 197, PF-2341066, NK4, XL-880, MP 470, K252a, c-MET blocking antibody, and combinations thereof.
3. The method of paragraph 1 or 2, wherein the cMET inhibitor is SU112274.
4. The method of paragraph 1, wherein the subject is a mammal.
5. The method of any paragraph 1-4, wherein the subject is a human.
6. The method of any paragraph 1-4, wherein the subject is a feline.
7. A method for treatment of polycystic kidney disease comprising:
   a) selecting a subject having polycystic kidney disease; and
   b) administering to a subject selected in step a) an effective amount of a pharmaceutical composition comprising a cMET inhibitor.
8. The method of paragraph 7, wherein the cMET inhibitor is selected from the group consisting of SU112274, PHA665752, ARQ 197, PF-2341066, NK4, XL-880, MP 470, K252a, c-MET blocking antibody, and combinations thereof.
9. The method of paragraph 7 or 8, wherein the cMET inhibitor is SU112274.
10. The method of any paragraph 1-9, wherein the subject is a mammal.
11. The method of any paragraph 1-10, wherein the subject is a human.
12. The method of any paragraph 1-10, wherein the subject is a feline.
13. The method of paragraph 2, wherein the cMET inhibitor is c-MET blocking antibody.
14. The method of paragraph 7 or 8, wherein the cMET inhibitor is c-MET blocking antibody.
15. A cMET inhibitor or a pharmaceutical salt thereof for use as a medicament for treating polycystic kidney disease in a subject in need thereof.
16. A cMET inhibitor or a pharmaceutical salt thereof selected from the group consisting of SU112274, PHA665752, ARQ 197, PF-2341066, NK4, XL-880, MP 470, K252a, c-MET blocking antibody, and combinations thereof for use as a medicament for treating polycystic kidney disease in a subject in need thereof.
17. SU112274 for use as a medicament for treating polycystic kidney disease in a subject in need thereof.
18. Use of a cMET inhibitor or a pharmaceutical salt thereof in the manufacture of a medicament for treating polycystic kidney disease in a subject in need thereof.
19. Use of a cMET inhibitor or a pharmaceutical salt thereof selected from the group consisting of SU112274, PHA665752, ARQ 197, PF-2341066, NK4, XL-880, MP 470, K252a, c-MET blocking antibody, and combinations thereof in the manufacture of a medicament for treating polycystic kidney disease in a subject in need thereof.
20. Use of SU112274 in the manufacture of a medicament for treating polycystic kidney disease in a subject in need thereof.
21. The use of any of claims 15-20, wherein the subject is a mammal.
22. The use of any of claims 15-21, wherein the subject is a human.
23. The use of any of claims 15-21, wherein the subject is a feline.

### EXAMPLES

### Example 1

### Reagents

Antibodies: rabbit polyclonal anti-mouse α3 integrin (Invitrogen #E0524804K, Carlsbad, Calif.), rabbit polyclonal anti-mouse mTOR and anti-mouse phospho-mTOR (Cell Signaling #2972, and #2971, Danvers, Mass.), mouse monoclonal anti-mouse c-MET (Cell Signaling #3127), mouse monoclonal anti-mouse Ubiquitin (Cell Signaling #3936), rabbit polyclonal anti-mouse c-Cb1 (Santa Cruz Biotechnology #sc-170, Santa Cruz, Calif.), mouse monoclonal anti-mouse GM130 (BD Biosciences #610822, San Jose, Calif.). Unless otherwise stated, all chemicals were purchased from Sigma.

### Example 2

### Cells and Mice

Pkd1^{null/null} mice are Pkd1 null mice, resulted in a null Pkd1 phenotype. Pkd1 wild type (WT) and Pkd1^{null/null} cell line were isolated from embryonic day 15.5 kidneys from a cross of Pkd1^{null/+} mice that also carry a temperature-sensitive simian virus 40 (SV40) large T-antigen transgene, similar with the protocol described in reference 23. Wt and Pkd1^{null/null} cells were cultured in Dulbecco's modified Eagle medium containing 2% fetal bovine serum, 0.75 μg/L γ-interferon, 1.0 g/L insulin, 0.67 mg/L sodium selenite, 0.55 g/L transferrin, 36 ng/ml hydrocortisone, 100 U/ml Penicillin/streptomycin under 33° C. and 5% CO₂²³.

### Example 3

### Immunoprecipitation and Western Blot

Immunoprecipitation and western-blot were performed using whole cell lysates unless otherwise specified. Confluent cells were collected, washed with PBS, lysed with lysis buffer (20 mM Tris/HCl, 1 mM EDTA, 150 mM NaCl, 1% Triton X-100) containing proteinase inhibitor cocktail tablet (Roche #1697498, Mannheim, Germany) at 4° C. for 30 minutes. After centrifugation at 13,000 rpm for 15 minutes, supernatants were incubated with specific antibody at 4° C. for 1 hour, followed by incubation with Protein G conjugated beads (Pierce Biotechnology, IL) at 4° C. for 2 hours, washed in lysis buffer. Samples were running on 7.5% acrylamide gel, transferred to PVDF membranes and visualized by immunoblotting with respective antibodies.

### Example 4

### Immunocytochemistry and Immunohistochemistry

Cultured cells or cryosections (embedded in OCT and cut in a thickness of 5 μm) were fixed in cold methanol at −20° C. for 10 minutes, blocked in 2% BSA for 1 hour, incubated overnight at 4° C. with primary antibody and then with Alex Fluor 488 or Alex Fluor 594 labeled secondary antibody at room temperature for 1 hour. Images were taken with the same exposure time for the same antibody.

### Example 5

### mTOR Phosphorylation

Pkd1^{null/null} and wild type cells were treated with either Hepatocyte Growth Factor (HGF, Sigma-Aldrich, St. Louis, Mo., 50 ng/ml. 20 minutes) or Met Kinase Inhibitor (5 μM, 4 hours, Calbiochem, La Jolla, Calif.). Blotting with phospho-mTOR antibody and total mTOR antibody was used to analyze mTOR phosphorylation.

### Example 6

### c-MET Degradation

Both wild type cells, Pkd1^{null/null} cells and α3 integrin−/− cells were stimulated with 50 ng/ml HGF for 30 minutes, cells were lysed and followed by western blot to show the c-MET amount, normalized with GAPDH. Band density was measured by densitometry (Gel Doc XR, Bio-Rad Laboratories Inc, Hercules, Calif.), according to the instructions of the manufacturer's manual.

### Example 7

### Sequential Precipitation with Avidin and α3 Integrin Antibody

Confluent wild type and Pkd1^{null/null} cells were labeled with membrane impermeable EZ-Link Sulfo-NHS-Biotin. Avidin conjugated beads were used to pull down labeled proteins. Unlabelled α3β1 integrin in the supernatant was immunoprecipitated with the polyclonal anti-α3 integrin antibody.

### Example 8

### Isolation of Golgi Apparatus Fraction

Isolation of Golgi fraction from cultured wild type and Pkd1^{null/null} cells was done by using a discontinuous sucrose gradient ultracentrifugation described by Balch et al⁴⁹. Briefly, confluent cells were harvested and washed in Homogenization Medium (10 mM Tris/HCl, pH 7.4, 250 mM sucrose) 2 times, homogenized in 3 ml Homogenization Medium, adjusted sucrose concentration to 1.4 M. Transfer 3.9 ml sample solution to an ˜11 ml ultracentrifuge tube, overlay sample with 3.9 ml of 1.2 M sucrose gradient solution and then 1.95 ml of 0.8 M sucrose gradient solution. Use a syringe to underlay sample with 1.3 ml of 1.6 M sucrose gradient solution. Centrifugation was carried out at 4° C., 110,000 g for 2 hours. The Golgi fraction band was harvested from the 0.8 M/1.2 M sucrose interface.

### Example 9

### Ubiquitination Analysis

Wild type and α3 integrin knockout cells were starved for 24 hours before being stimulated by HGF at 50 ng/ml for 10 minutes. Cells were collected after HGF stimulation, immunoprecipitated with c-MET antibody (Cell Signaling, MA) and blotted with ubiquitin antibody (1:1000, mouse monoclonal, Cell Signaling). Controls include cell lysates from wild type and α3 integrin knockout cells without HGF stimulation.

### Example 10

### Real-time PCR

Real-time PCR was carried out on Smart Cycler II. SyBR Green was used for fluorescence detection. PCR parameters: 95° C., 10 minutes, (95° C., 15 seconds, 60° C., 30 seconds, 72° C., 30 seconds) 40 cycles, melting temperature measured between 60-95° C. c-MET forward primer: ACG GCT GAA GGA AAC CCA AG, reverse primer: ACC CAG AGT CTA CGG AAC AGA. c-MET mRNA amount was normalized by 18S RNA amount from the same cDNA sample.

### Example 11

### Glycosylation Analysis

Wild type and Pkd1^{null/null} cells were lysed and incubated with Endo-H and PNGase F glycosidase enzymes (New England Biolabs, MA), following the manufacturer's manual for the digestion. Western blot under reducing conditions with antibody against C-terminal of α3 integrin was used to evaluate the migration change before and after Endo-H and PNGase F digestion.

### Example 12

### Organ Culture in vitro

Embryonic mice kidneys of E13.5 were dissected out and cultured in media²⁹ (1% FBS, 5 mg/ml Transferin, 0.05 mM Sodium Selenite, 100 nM hydrocortisone, 2 nM T3, 25 ng/ml PGE1, 100 U/ml Penicillin/streptomycin, 100 mM 8-Br-cAMP) in Center-Well Organ Culture Dish (BD Labware, Franklin Lakes, N.J.). The following day, the kidneys from the same embryo were treated with either 5 mM Met Kinase Inhibitor (Calbiochem, La Jolla, CA) or DMSO (the same volume with Met Kinase Inhibitor). The media were changed everyday with the same additives as above. After 5 days, kidneys were fixed by 4% PFA, and embedded in paraffin. Paraffin sections were stained with hematoxylin and eosin.

### Example 13

### Cystogenesis Inhibition After Blocking c-MET Signaling Pathway

Embryonic mice kidneys of E13.5 were dissected out and cultured in media (1% FBS, 5 mg/ml Transferin, 0.05 mM Sodium Seelenite, 100 nM hydrocortisone, 2 nM T3, 25ng/ml PGE1, 100 U/ml Penicillin/streptomycin, 100 mM 8-Br-cAMP) in Center-Well Organ Culture Dish (BD Labware, Franklin Lakes, N.J.). The following day, the kidneys from the same embryo were treated with either 2 microgram/ml c-MET blocking antibody (R&D Systems, Minneapolis, Minn.) or PBS (the same volume with c-Met blocking antibody). The media were changed everyday with the same additives as above. After 4 days, kidneys were fixed by 4% PFA, and embedded in paraffin. Paraffin sections were stained with hematoxylin and eosin.

Here we show that glycosylation of the α3 integrin subunit is defective, and α3β1 integrin is retained in the Golgi apparatus in Pkd1^{null/null} cells, a Pkd1 null mutant cell line²³. c-Cb1, an E3 ubiquitin ligase normally responsible for ubiquitination of c-MET, is also sequestered in the Golgi apparatus with α3β1 integrin in Pkd1^{null/null} cells. Consistent with these results, ubiquitination of c-MET after stimulation with HGF is defective in Pkd1^{null/null} cells and there is an increased c-MET dependent activation of the PI3K/Akt/mTOR signaling pathway. Additionally, pharmacological blockade of c-MET signaling results in a dramatic decrease in cyst formation in an organ culture model of PKD.

### Example 14

### c-Met Antagonists Ameliorate the Cyst Formations in Kidneys

E13.5 embryonic kidneys were put in organ culture along with 100 μM 8-Br-cAMP, with or without 5 μM c-MET inhibitor (SU11274, from Calbiochem). (B) E13.5 embryonic kidneys were put in organ culture along with 100 mM 8-Br-cAMP, with or without 5 μg/ml c-MET neutralizing antibody (R&D Systems). (C) E13.5 embryonic kidneys were put in organ culture along with 100 mM 8-Br-cAMP, with or without 0.5 μM c-MET inhibitor (PHA665752, from Tocris, UK). Hematoxylin & Eosin stained sections of kidneys are shown after 96 hours of culture. Cyst formation was decreased by c-MET inhibitor or c-MET neutralizing antibody treatment in the Pkd1 null/null kidneys, with no apparent effect on nephrogenesis.

### Example 15

### c-Met inhibitor to treat Pkd+/− Pregnant Mice

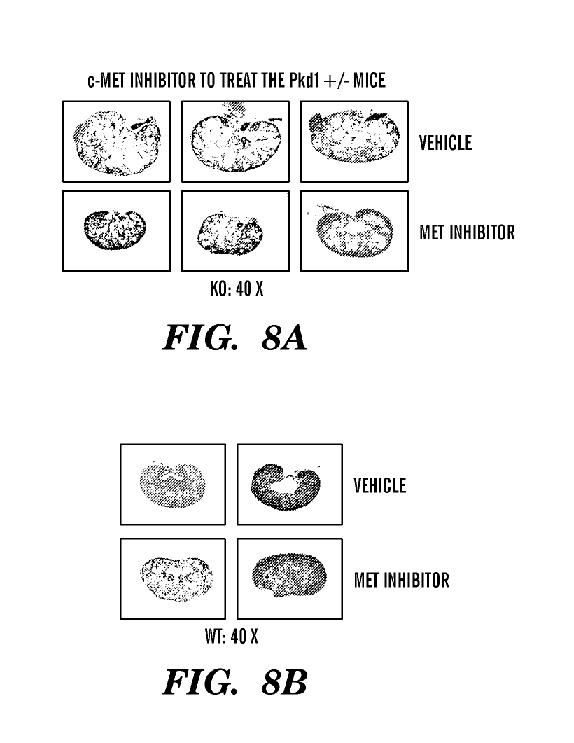

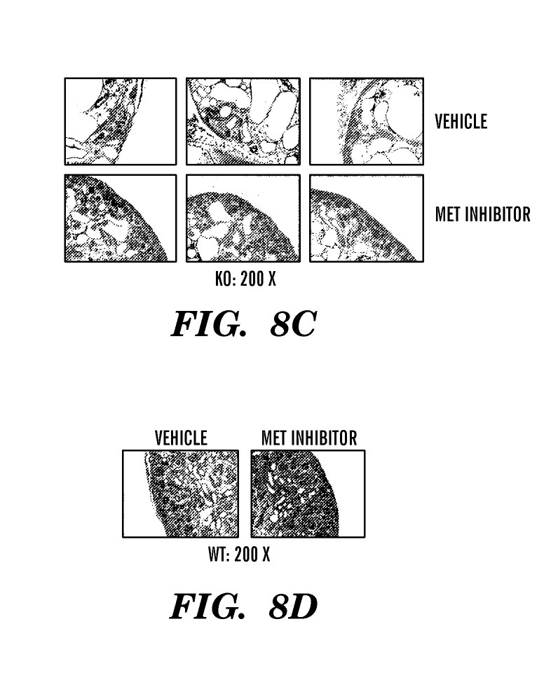

Male and female Pkd1 +/− mice were inter-crossed to obtain homozygous mutant embryos. At E14.5, pregnant Pkd1 +/− females received intraperitoneally injections of either c-MET kinase inhibitor (Calbiochem, #448101) or vehicle. c-MET kinase inhibitor was dissolved in 30% DMSO/20Ethanol/50% PBS (vehicle), and injected at a amount of 100 mg/kg/day, divided into 2 doses, 1 dose in the morning and the other dose in the evening. The pregnant mice were injected at E14.5, E15.5, E16.5, and E17.5, and sacrificed at E18.5. The E18.5 embryonic kidneys were fixed in 4% PFA, genotyped, and paraffin sections were obtained and stained with Hematoxylin and Eosin. See FIG. 8.

### Results

### Hyperactivation of mTOR in Pkd1^{null/null} Cells is Dependent on c-MET

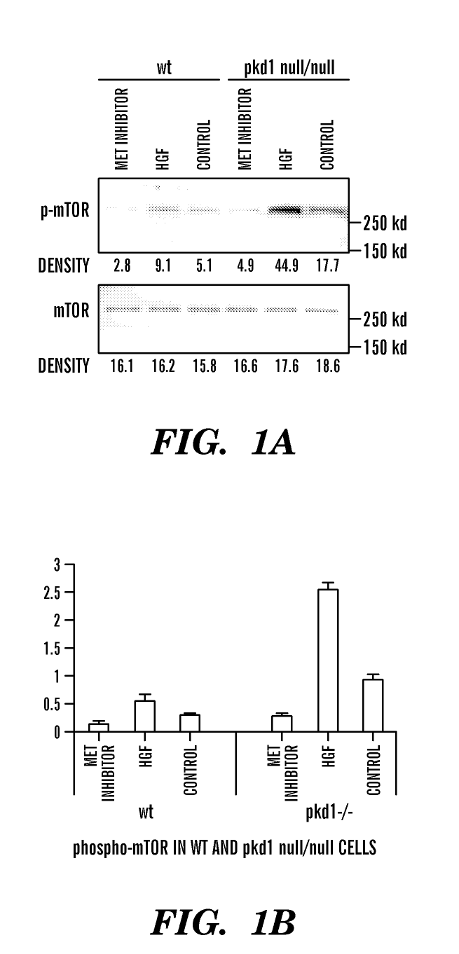

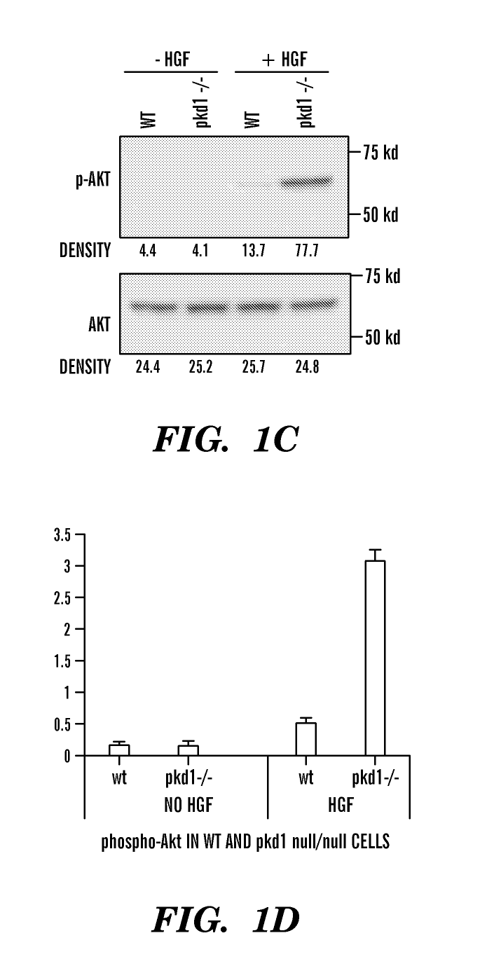

Consistent with previously published results, mTOR was hyperphosphorylated in Pkd1^{null/null} cells²². Stimulation with HGF accentuated the difference in mTOR phosphorylation between Pkd1^{null/null} and wild type (WT) cells, whereas treatment with a c-MET inhibitor (Met Kinase Inhibitor, Calbiochem) reduced mTOR phosphorylation in Pkd1^{null/null} cells, to a baseline level observed in WT cells (FIG. 1a, b). HGF-dependent phosphorylation of Akt was also greater in Pkd1^{null/null} cells than that in WT cells (FIG. 1c, d). These results indicate that hyperactivation of mTOR in polycystic kidney disease may occur downstream of the receptor tyrosine kinase c-MET.

### Defective Ubiquitination of c-MET in Pkd1^{null/null} Cells

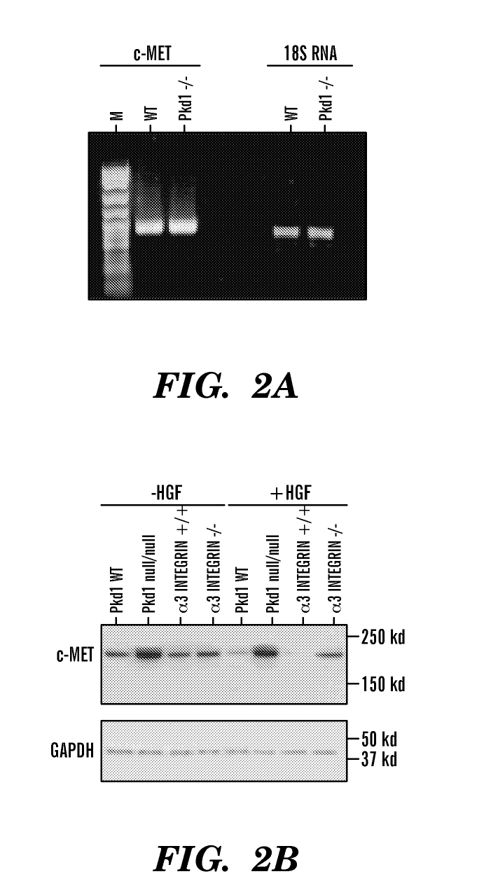

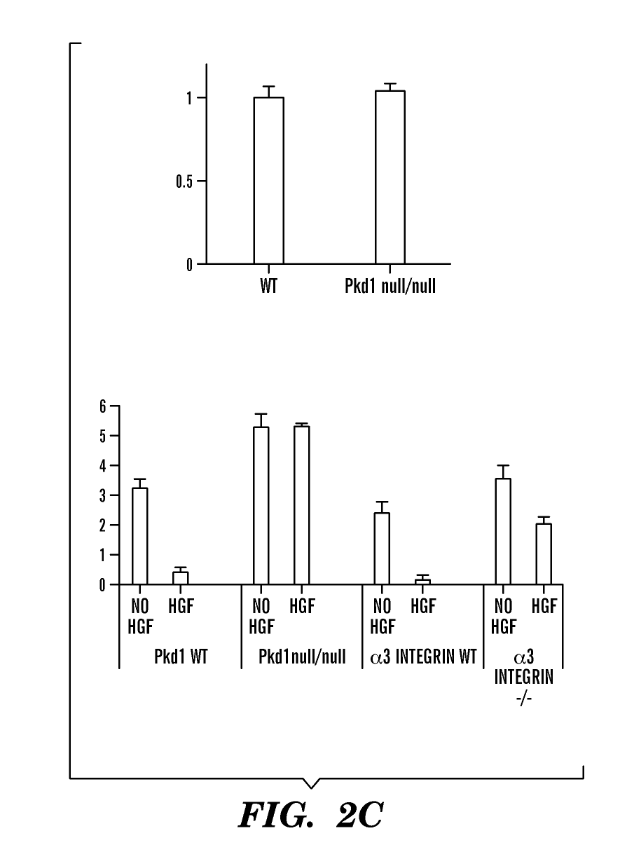

To elucidate the mechanism whereby HGF stimulation resulted in hyperphosphorylation of mTOR in Pkd1^{null/null} cells, we first examined levels of c-MET, Akt and mTOR in Pkd1^{null/null} and WT cells. Akt and mTOR were present at equivalent levels (FIG. 1a,c), whereas c-MET was more abundant in Pkd1^{null/null} cells (FIG. 2b). Increased protein levels of c-MET could reflect either increased synthesis or defective degradation of the protein. No difference in c-MET mRNA levels was observed between WT and Pkd1^{null/null} cells (FIG. 2a,b). Translational control of c-MET expression has not yet been examined. However, a marked difference in post-stimulatory degradation of c-MET was observed: 30 minutes after HGF stimulation of serum-starved cells, the level of c-MET was reduced 6-fold in WT cells, but negligibly reduced in Pkd1^{null/null} cells, relative to the pre-stimulatory level of c-MET in each cell type (FIG. 2c).

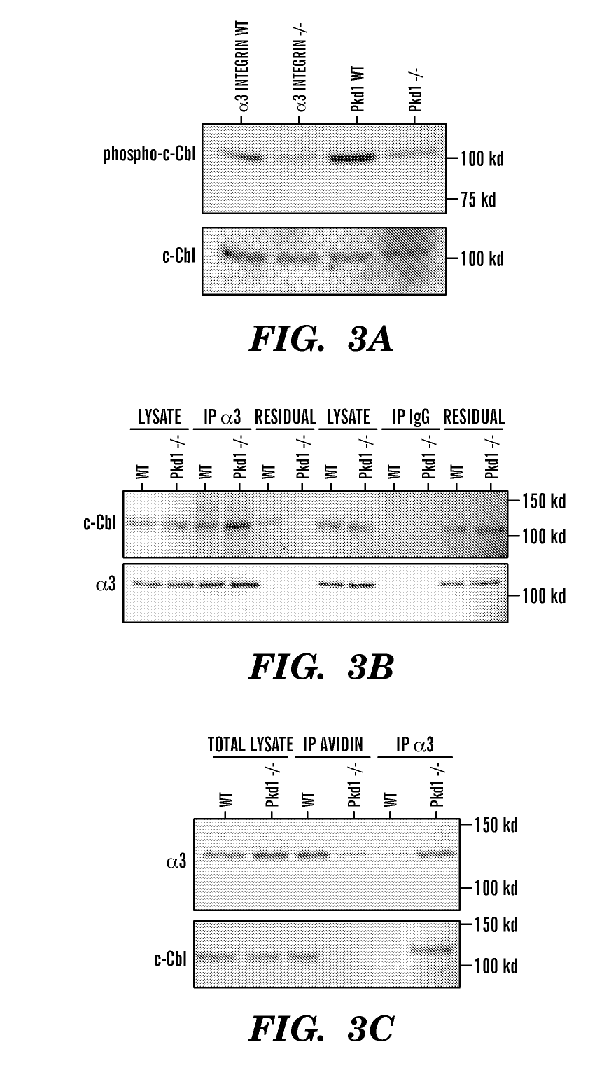

Degradation of c-MET occurs through two distinct pathways. One pathway is ligand-dependent through ubiquitination, the other is ligand-independent through shedding of an extracellular domain^{24,25}. Because our observed difference in c-MET reflected a post-stimulatory situation, we examined ubiquitination of c-MET. Abundant ubiquitination of c-MET after HGF stimulation was apparent in WT cells but virtually undetectable in Pkd1^{null/null} cells (FIG. 2c). Ubiquitination of c-MET requires association of the c-MET cytoplasmic domain with the c-Cb1 E3 ubiquitin ligase, and subsequent phosphorylation of c-Cb1. Phosphorylation of c-Cb1 after HGF stimulation was decreased in Pkd1^{null/null} cells compared with that in WT cells (FIG. 3a). Thus, the absence of polycystin-1 appeared to dramatically affect ubiquitination of c-MET through c-Cb1.

### Sequestration of α3β1 integrin and c-Cb1 in the Golgi Apparatus in Pkd1^{null/null} Cells

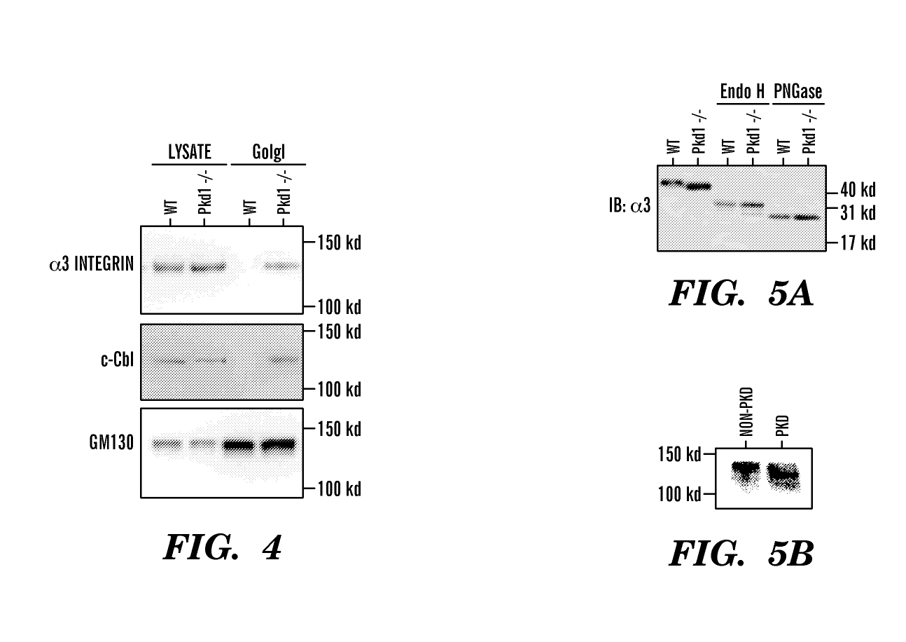

α3β1 integrin is highly expressed by WT and Pkd1^{null/null} cells. As c-Cb1 is known to interact with integrins²⁶, the role of α3β1 integrin on c-Cb1 phosphorylation and localization was examined. In α3 integrin −/− cells²⁷, c-Cb1 phosphorylation after HGF stimulation was decreased compared with α3 integrin +/+ cells (FIG. 3a), demonstrating that maximal c-Cb1 phosphorylation requires the presence of α3β1 integrin. Co-immunoprecipitation demonstrated nearly complete association of c-Cb1 with α3β1 integrin in both WT and Pkd1^{null/null} cells, little or no c-Cb1 was found in residual extracts after immuno-depletion of α3β1 integrin (FIG. 3c). However, while co-staining of α3β1 integrin and c-Cb1 in WT cells demonstrated membrane co-localization along cell-cell junctions (data not shown), both α3β1 integrin and c-Cb1 appeared to have acquired a Golgi apparatus localization in Pkd1^{null/null} cells (data not shown). This was confirmed by co-staining with the Golgi marker GM130, staining of which only overlapped with c-Cb1 and α3β1 integrin in Pkd1^{null/null} cells (data not shown). Additionally, biotinylation of cell surface proteins followed by affinity purification with immobilized Neutravidin protein beads confirmed the decreased membrane localization of α3β1 integrin and the cytoplasmic association of α3β1 integrin with c-Cb1 in Pkd1^{null/null} cells (FIG. 3b). When discontinuous sucrose gradient separation was used to enrich a Golgi apparatus fraction, α3β1 integrin and c-Cb1 were found in the Golgi apparatus-enriched fraction of Pkd1^{null/null} cells but not WT cells (FIG. 4).

The association of c-Cb1 with α3β1 integrin prompted us to examine c-MET degradation in the absence of α3β1 integrin. Equivalent c-MET (FIG. 2B) and c-Cb1 (FIG. 3a) were present in wild type and α3 integrin-deficient cells. After HGF stimulation, c-MET was incompletely degraded in α3 integrin −/− cells (FIG. 2b), demonstrating that c-MET ubiquitination after HGF stimulation in epithelial cells requires the presence of α3β1 integrin. Thus, α3β1 integrin may be involved in localizing c-Cb1 at the plasma membrane as part of a complex that regulates signaling by c-MET.

Together, these results demonstrate that in the absence of polycystin-1, α3β1 integrin appears to sequester c-Cb1 in the Golgi apparatus, and in so doing, limits the ability of cells to attenuate signaling by c-MET. This, in turn, hyperactivates mTOR, with its resultant effect on cell behavior, which is thought to lead to cystogenesis.

### Glycosylaton of α3 Integrin Subunit is Defective in Pkd1^{null/null} Cells

The finding that α3β1 integrin and c-Cb1 were mislocalized in the Golgi in Pkd1^{null/null} cells was reminiscent of findings that E-cadherin is also improperly processed in the Golgi apparatus in Pkd1^{null/null} cells²⁸. Since the modification of protein glycosylation is a major event occurring in the Golgi, the glycosylation of the α3 subunit was examined. We observed that the α3 integrin subunit displayed an abnormal mobility in SDS-PAGE electrophoresis (FIG. 5). Moreover, treatment of the cell lysate with alkaline phosphatase did not eliminate this difference in mobility, discounting the possibility of differential protein phosphorylation between WT and Pkd1^{null/null} cells. In contrast, treatment with PNGase F and Endo H eliminated the difference in mobility (FIG. 5), and comparison of the migration after treatment with PNGase F vs. Endo H suggested that high mannose modification is normal in Pkd1^{null/null} cells, while more complex glycosylation steps may be defective, a result more consistent with a defect in glycosylation that occurs in the Golgi apparatus.

### Altered Distribution of c-Cb1 and α3β1 Integrin in vivo in Pkd1^{null/null} Kidneys

To confirm that our findings with immortalized cell lines were relevant to changes that occurred in vivo, we compared the localization of α3β1 integrin and c-Cb1 in epithelial cells of WT and Pkd1^{null/null} kidneys. As predicted, both α3β1 integrin and c-Cb1 showed a basolateral distribution in tubules of WT kidneys (data not shown). In contrast, and reflecting the in vitro observations, in epithelial cells lining the cysts of Pkd1^{null/null} kidneys, both c-Cb1 and α3β1 integrin localized in a perinuclear distribution(data not shown). These in vivo data confirmed that c-Cb1 and α3β1 integrin are sequestered in the cytoplasm of epithelial cells, and cannot be correctly targeted to the plasma membrane.

### Treatment of Pkd1^{null/null} Cystic Kidneys in Organ Culture with c-MET Inhibitor can Decrease the Size and Number of Kidney Cysts

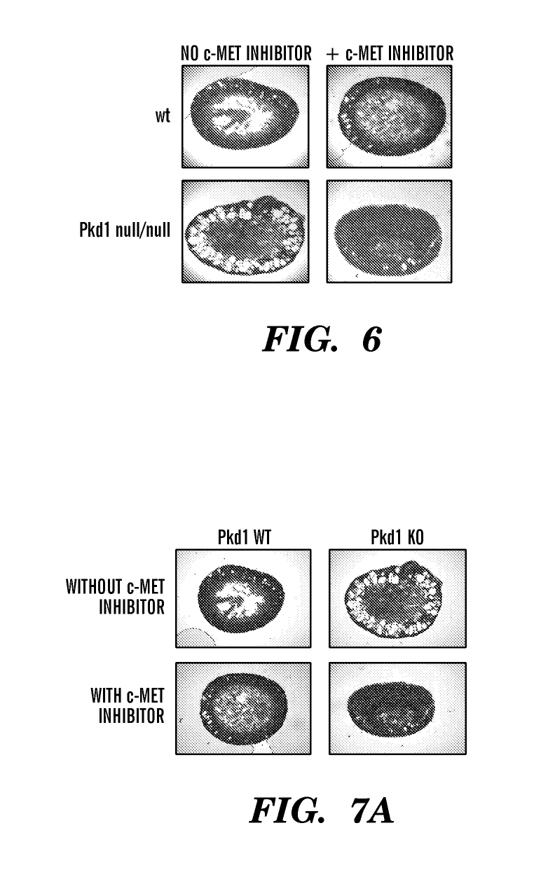

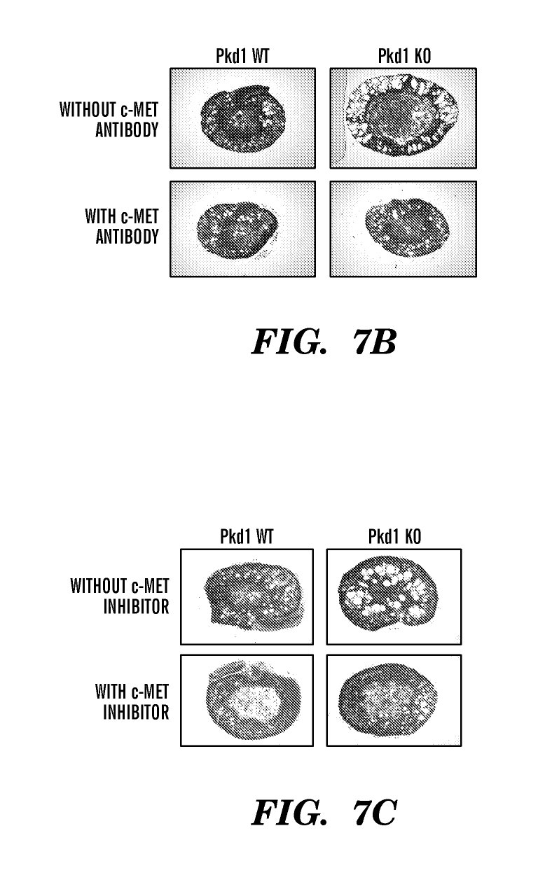

These observations predict that blockade of signaling by c-MET would reduce cyst formation in Pkd1^{null/null} kidneys. As a first test of this hypothesis, embryonic organ culture from WT and Pkd1^{null/null} kidneys were treated with a pharmacological blocker of c-MET. Typically, kidneys placed in organ culture, even from Pkd1−/− mice, do not develop or maintain cysts unless treated with 8-Br-cAMP²⁹, a cell permeable cAMP analog that is more resistant to phosphodiesterase cleavage than cAMP and that preferentially activates cAMP-dependent protein kinase (PKA)^{30,31}. An appropriate concentration of 8-Br-cAMP was used that promoted more cyst formation in Pkd1^{null/null} kidneys than in WT kidneys. Treatment with the c-MET inhibitor reduced cyst formation in organ culture by Pkd1^{null/null} mutant kidneys to the basal level observed in WT kidneys (FIG. 6). Importantly, the c-Met inhibitor did not have a marked effect on nephrogenesis in either wild type or mutant kidneys (FIG. 6).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

One skilled in the art would also readily appreciate that the present invention is well adapted to carry out the objects and obtain the ends and advantages mentioned, as well as those inherent herein. The molecular complexes and the methods, procedures, treatments, molecules, specific compounds described herein are presently representative of preferred embodiments, are exemplary, and are not intended as limitations on the scope of the invention. Changes therein and other uses will occur to those skilled in the art which are encompassed within the spirit of the invention are defined by the scope of the claims.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### REFERENCES

1. Lu, W. et al. Late onset of renal and hepatic cysts in Pkd1-targeted heterozygotes. Nat Genet 21, 160-1 (1999).

2. Mochizuki, T. et al. PKD2, a gene for polycystic kidney disease that encodes an integral membrane protein. Science 272, 1339-42 (1996).

3. Igarashi, P. & Somlo, S. Polycystic kidney disease. J Am Soc Nephrol 18, 1371-3 (2007).

4. Nakamura, T. et al. Growth factor gene expression in kidney of murine polycystic kidney disease. J Am Soc Nephrol 3, 1378-86 (1993).

5. Matsell, D. G. et al. Insulin-like growth factor (IGF) and IGF binding protein gene expression in multicystic renal dysplasia. J Am Soc Nephrol 8, 85-94 (1997).

6. Horie, S. et al. Mediation of renal cyst formation by hepatocyte growth factor. Lancet 344, 789-91 (1994).

7. Sullivan, L. P. et al. Sulfonylurea-sensitive K(+) transport is involved in Cl(−) secretion and cyst growth by cultured ADPKD cells. J Am Soc Nephrol 13, 2619-27 (2002).

8. Leipziger, J. Control of epithelial transport via luminal P2 receptors. Am J Physiol Renal Physiol 284, F419-32 (2003).

9. Joly, D. et al. Laminin 5 regulates polycystic kidney cell proliferation and cyst formation. J Biol Chem 281, 29181-9 (2006).

10. Malhas, A. N., Abuknesha, R. A. & Price, R. G. Interaction of the leucine-rich repeats of polycystin-1 with extracellular matrix proteins: possible role in cell proliferation. J Am Soc Nephrol 13, 19-26 (2002).

11. Kovacs, J. et al. Differential growth factor-induced modulation of proteoglycans synthesized by normal human renal versus cyst-derived cells. J Am Soc Nephrol 5, 47-54 (1994).

12. Pachnis, V., Mankoo, B. & Costantini, F. Expression of the c-ret proto-oncogene during mouse embryogenesis. Development 119, 1005-17 (1993).

13. Dudley, A. T., Godin, R. E. & Robertson, E. J. Interaction between FGF and BMP signaling pathways regulates development of metanephric mesenchyme. Genes Dev 13, 1601-13 (1999).

14. Kuo, N. T., Norman, J. T. & Wilson, P. D. Acidic FGF regulation of hyperproliferation of fibroblasts in human autosomal dominant polycystic kidney disease. Biochem Mol Med 61, 178-91 (1997).

15. Santos, O. F. & Nigam, S. K. HGF-induced tubulogenesis and branching of epithelial cells is modulated by extracellular matrix and TGF-beta. Dev Biol 160, 293-302 (1993).

16. Santos, O. F. et al. Involvement of hepatocyte growth factor in kidney development. Dev Biol 163, 525-9 (1994).

17. Konda, R. et al. Expression of hepatocyte growth factor and its receptor C-met in acquired renal cystic disease associated with renal cell carcinoma. J Urol 171, 2166-70 (2004).

18. Kreidberg, J. A. et al. Alpha 3 beta 1 integrin has a crucial role in kidney and lung organogenesis. Development 122, 3537-47 (1996).

19. Shannon, M. B., Patton, B. L., Harvey, S. J. & Miner, J. H. A hypomorphic mutation in the mouse laminin alpha5 gene causes polycystic kidney disease. J Am Soc Nephrol 17, 1913-22 (2006).

20. Chang, J. C., Chang, H. H., Lin, C. T. & Lo, S. J. The integrin alpha6betal modulation of PI3K and Cdc42 activities induces dynamic filopodium formation in human platelets. J Biomed Sci 12, 881-98 (2005).

21. Tang, K., Nie, D., Cai, Y. & Honn, K. V. The beta4 integrin subunit rescues A431 cells from apoptosis through a PI3K/Akt kinase signaling pathway. Biochem Biophys Res Commun 264, 127-32 (1999).

22. Shillingford, J. M. et al. The mTOR pathway is regulated by polycystin-1, and its inhibition reverses renal cystogenesis in polycystic kidney disease. Proc Natl Acad Sci USA 103, 5466-71 (2006).

23. Nauli, S. M. et al. Polycystins 1 and 2 mediate mechanosensation in the primary cilium of kidney cells. Nat Genet 33, 129-37 (2003).

24. Abella, J. V. et al. Met/Hepatocyte growth factor receptor ubiquitination suppresses transformation and is required for Hrs phosphorylation. Mol Cell Biol 25, 9632-45 (2005).

25. Nath, D., Williamson, N.J., Jarvis, R. & Murphy, G. Shedding of c-Met is regulated by crosstalk between a G-protein coupled receptor and the EGF receptor and is mediated by a TIMP-3 sensitive metalloproteinase. J Cell Sci 114, 1213-20 (2001).

26. Kaabeche, K. et al. Cb1-mediated ubiquitination of alpha5 integrin subunit mediates fibronectin-dependent osteoblast detachment and apoptosis induced by FGFR2 activation. J Cell Sci 118, 1223-32 (2005).

27. Wang, Z. et al. (Alpha)3(beta)1 integrin regulates epithelial cytoskeletal organization. J Cell Sci 112 (Pt 17), 2925-35 (1999).

28. Charron, A. J., Nakamura, S., Bacallao, R. & Wandinger-Ness, A. Compromised cytoarchitecture and polarized trafficking in autosomal dominant polycystic kidney disease cells. J Cell Biol 149, 111-24 (2000).

29. Magenheimer, B. S. et al. Early embryonic renal tubules of wild-type and polycystic kidney disease kidneys respond to cAMP stimulation with cystic fibrosis transmembrane conductance regulator/Na(+),K(+),2C1(−) Co-transporter-dependent cystic dilation. J Am Soc Nephrol 17, 3424-37 (2006).

30. Meyer, R. B., Jr. & Miller, J.P. Analogs of cyclic AMP and cyclic GMP: general methods of synthesis and the relationship of structure to enzymic activity. Life Sci 14, 1019-40 (1974).

31. Kasagi, Y., Horiba, N., Sakai, K., Fukuda, Y. & Suda, T. Involvement of cAMP-response element binding protein in corticotropin-releasing factor (CRF)-induced down-regulation of CRF receptor 1 gene expression in rat anterior pituitary cells. J Neuroendocrinol 14, 587-92 (2002).

32. Yu, W. et al. Hepatocyte growth factor switches orientation of polarity and mode of movement during morphogenesis of multicellular epithelial structures. Mol Biol Cell 14, 748-63 (2003).

33. Kamei, T. et al. Coendocytosis of cadherin and c-Met coupled to disruption of cell-cell adhesion in MDCK cells—regulation by Rho, Rac and Rab small G proteins. Oncogene 18, 6776-84 (1999).

34. Takayama, H., LaRochelle, W. J., Sabnis, S. G., Otsuka, T. & Merlino, G. Renal tubular hyperplasia, polycystic disease, and glomerulosclerosis in transgenic mice overexpres sing hepatocyte growth factor/scatter factor. Lab Invest 77, 131-8 (1997).

35. Lelongt, B., Carone, F. A. & Kanwar, Y. S. Decreased de novo synthesis of proteoglycans in drug-induced renal cystic disease. Proc Natl Acad Sci USA 85, 9047-51 (1988).

36. Jin, H., Carone, F. A., Nakamura, S., Liu, Z. Z. & Kanwar, Y. S. Altered synthesis and intracellular transport of proteoglycans by cyst-derived cells from human polycystic kidneys. J Am Soc Nephrol 2, 1726-33 (1992).

37. Ehara, T., Carone, F. A., McCarthy, K. J. & Couchman, J. R. Basement membrane chondroitin sulfate proteoglycan alterations in a rat model of polycystic kidney disease. Am J Pathol 144, 612-21 (1994).

38. Beavan, L. A. et al. Comparison of proteoglycans synthesized by porcine normal and polycystic renal tubular epithelial cells in vitro. Arch Biochem Biophys 284, 392-9 (1991).

39. Yoder, B. K. Role of primary cilia in the pathogenesis of polycystic kidney disease. J Am Soc Nephrol 18, 1381-8 (2007).

40. Yoder, B. K., Hou, X. & Guay-Woodford, L. M. The polycystic kidney disease proteins, polycystin-1, polycystin-2, polaris, and cystin, are co-localized in renal cilia. J Am Soc Nephrol 13, 2508-16 (2002).

41. Charron, A. J., Bacallao, R. L. & Wandinger-Ness, A. ADPKD: a human disease altering Golgi function and basolateral exocytosis in renal epithelia. Traffic 1, 675-86 (2000).

42. Poole, C. A., Zhang, Z. J. & Ross, J. M. The differential distribution of acetylated and detyrosinated alpha-tubulin in the microtubular cytoskeleton and primary cilia of hyaline cartilage chondrocytes. J Anat 199, 393-405 (2001).

43. Follit, J. A., Tuft, R. A., Fogarty, K. E. & Pazour, G. J. The intraflagellar transport protein IFT20 is associated with the Golgi complex and is required for cilia assembly. Mol Biol Cell 17, 3781-92 (2006).

44. Tenkova, T. & Chaldakov, G. N. Golgi-cilium complex in rabbit ciliary process cells. Cell Struct Funct 13, 455-8 (1988).

45. Girod, C., Lheritier, M. & Guichard, Y. (Cilia-centriole-Golgi apparatus relationships in the glandular cells of the anterior pituitary of the hedgehog (Erinaceus europaeus L)). C R Seances Acad Sci D 290, 711-4 (1980).

46. Yoshimura, S., Egerer, J., Fuchs, E., Haas, A. K. & Barr, F. A. Functional dissection of Rab GTPases involved in primary cilium formation. J Cell Biol 178, 363-9 (2007).

47. Stephens, R. E. Ciliary protein turnover continues in the presence of inhibitors of golgi function: evidence for membrane protein pools and unconventional intracellular membrane dynamics. J Exp Zool 289, 335-49 (2001).

48. Kleymenova, E. et al. Tuberin-dependent membrane localization of polycystin-1: a functional link between polycystic kidney disease and the TSC2 tumor suppressor gene. Mol Cell 7, 823-32 (2001).

49. Balch, W. E., Dunphy, W. G., Braell, W. A. & Rothman, J. E. Reconstitution of the transport of protein between successive compartments of the Golgi measured by the coupled incorporation of N-acetylglucosamine. Cell 39, 405-16 (1984).

All references herein are incorporated by reference in their entirety.

1. A method for treating polycystic kidney disease in a subject in need thereof which comprises providing to said subject an effective amount of a cMET inhibitor or a pharmaceutical salt thereof.

2. The method of claim 1, wherein the cMET inhibitor is selected from the group consisting of SU112274, PHA665752, ARQ 197, PF-2341066, NK4, XL-880, MP 470, K252a, c-MET blocking antibody, and combinations thereof.

3. The method of claim 2, wherein the cMET inhibitor is SU112274.

4. The method of claim 1, wherein the subject is a mammal.

5. The method of claim 4, wherein the subject is a human.

6. The method of claim 4, wherein the subject is a feline.

7. A method for treatment of polycystic kidney disease comprising:
a) selecting a subject having polycystic kidney disease; and
b) administering to a subject selected in step a) an effective amount of a pharmaceutical composition comprising a cMET inhibitor.

8. The method of claim 7, wherein the cMET inhibitor is selected from the group consisting of SU112274, PHA665752, ARQ 197, PF-2341066, NK4, XL-880, MP 470, K252a, c-MET blocking antibody, and combinations thereof.

9. The method of claim 8, wherein the cMET inhibitor is SU112274.

10. The method of claim 1, wherein the subject is a mammal.

11. The method of claim 10, wherein the subject is a human.

12. The method of claim 10, wherein the subject is a feline.

13. The method of claim 2, wherein the cMET inhibitor is c-MET blocking antibody.

14. The method of claim 8, wherein the cMET inhibitor is c-MET blocking antibody.

15. (canceled)

16. (canceled)

17. (canceled)

18. (canceled)

19. (canceled)

20. (canceled)

21. (canceled)

22. (canceled)

23. (canceled)
